(19) United States
(12) Patent Application Publication
(10) Pub. No.: US 2016/0022864 A1
(43) Pub. Date: Jan. 28, 2016

(54) Tiny Bone Defect Repairing Material, Matrix Material Thereof and Producing Method Thereof

(71) Applicants: Chia-Tze Kao, Taichung City (TW); Ming-You Shie, Kaohsiung City (TW)

(72) Inventors: Chia-Tze Kao, Taichung City (TW); Ming-You Shie, Kaohsiung City (TW)

(21) Appl. No.: 14/337,312

(22) Filed: Jul. 22, 2014

(51) Int. Cl. A61L 27/18; A61L 27/12

(52) U.S. Cl. CPC A61L 27/18; A61L 27/12; A61L 2430/02

(57) ABSTRACT

The present invention provides a producing method for a tiny bone defect repairing material. The invention solves the problems that the setting time is too long to cause bad mechanical property in conventional bone cements and also remains bioactivities and water absorb ability. The invention has no cytotoxicity and enables to stimulate cells growth.

### BACKGROUND OF THE INVENTION

1. Field of Invention

This invention relates to materials for repairing bone defects, specifically for an artificial material for repairing tiny human bone defects.

2. Description of the Related Art

Calcium silicate-based bone cement is widely used in clinical situations. Silicon (Si) is an important trace element in an early stage of bone formation. Silicon (Si) is able to stimulate bone tissue regeneration and to increase bone cells proliferation. Calcium silicate-base bone cement becomes a major biomedical material in repairing or reconstructing bone defects. Materials such as mineral trioxide aggregate (MTA) and bio-glass are most common calcium silicate-based bone cements in the market. Clinical setting time of the mineral trioxide is around 162 minutes or more that leading to worse injectability and plasticity. The bio-glass has bad mechanical property that only suitable for repairing particular bone damage for which the bone doesn't need to support too much pressure, such like ear ossicle and finger bones.

Some solutions were revealed that trying to reduce the setting time of the conventional calcium silicate-based bone cement. These improved conventional calcium silicate-based bone cement may cause a worse mechanical property, injectability and plasticity thereof. Some macromolecular materials such as gelatin, chitosan or collagen are added into the conventional calcium silicate-based bone cement to improve the said mechanical property. However, calcium silicate-bone cement with macromolecular materials may further increase setting time. Thus, the conventional calcium silicate-based bone cement has many aforementioned disadvantages that need to be solved.

### SUMMARY OF THE INVENTION

In order to solve the disadvantages and shortcomings of the conventional calcium silicate-based bone cement. The present invention provides a tiny bone defect repairing material and a producing method thereof to obviate or mitigate the shortcoming of the prior art.

The producing method of a tiny bone defect repairing material having steps of:
mixing a polydopamine solution with a calcium silicate-based material to form a polydopamine modified calcium silicate-based compound; and
blending a secondary water or a phosphate solution with the polydopamine modified calcium silicate-based compound to obtain the tiny bone defect repairing material.

Thus, the present invention achieves advantages as below.

1. Providing a tiny bone defect repairing material with high performance in abilities of injection, plasticity for clinical use. 2. The present invention provide high standard of mechanical property, bioactivity and clinical used property of the tiny bond defect repairing material. 3. The present invention provides improved bioactivity and water absorbs ability compared to the conventional calcium silicate-based bone cement.

### BRIEF DESCRIPTION OF THE DRAWINGS

FIG. 1 illustrates abilities of injection of the present invention in various blending ratio;

FIG. 2 illustrates setting time of the present invention in different blending ratio;

FIG. 3 is an XRD pattern of each testing block of the present invention;

FIG. 4 is surface SEM observation result of the present invention;

FIG. 5 is surface SEM observation result of the present invention after steeped in stimulated body fluid for 24 hours;

FIG. 6 is a bar diagram of cells attachment and growth on the present invention; and

FIG. 7 is a test result for phosphatase secretion of the primary human pulp fibroblast (HPF) growth on present invention.

### DETAILED DESCRIPTION OF THE INVENTION

A producing method of an artificial material for repairing tiny bone defects in accordance with the present invention has processing steps of:
mixing a polydopamine solution with a calcium silicate-based material. The calcium silicate-based material and the polydopamine solution are filtered to form a mixing powder. The mixing powder is washed by water which the water may be deionized by a distilled water to form a secondary water. The mixing powder is filtered again and dried to form a polydopamine modified calcium silicate-based compound. The polydopamine solution is prepared by dissolving a polydopamine powder into water or solvent to form a polydopamine liquid. The polydopamine liquid may further mixed with a Tris buffer solution 150 mL to form the polydopamine solution. The concentration of the polydopamine liquid is between 0.5˜20 mg/ml. A preferred concentration of the polydopamine liquid is between 0.5˜5.0 mg/ml. A pH value of the Tris buffer solution is 8.5. The temperature of the Tris buffer solution is 80° C. The calcium silicate-based material is stirred with the polydopamine solution by a mixing spoon. The polydopamine solution may modify or polymerize with the calcium silicate-based material. The Tris buffer solution is able to stable pH value of whole reaction and makes the calcium silicate-based material becomes more easily to polymerize with the polydopamine solution. The mixing powder is filtered by a filter paper and dried by an oven or the like equipment.
blending a secondary water or a phosphate solution with the polydopamine modified calcium silicate-based compound uniformly to form a tiny bone defect repairing material. Ratio of the secondary water or the phosphate solution to the polydopamine modified calcium silicate-based compound is between 0.2˜4 mL/g. The preferred ratio of the secondary water or the phosphate solution to the polydopamine modified calcium silicate-based compound is between 0.8˜1.5 mL/g. The phosphate solution may accelerate the setting time and the time of hydration reaction after blending the polydopamine modified calcium silicate-based compound with the water or the phosphate solution.

Aforementioned calcium silicate-based material may be a type I calcium silicate-based material or a type II calcium silicate-based material. Producing method of the type I calcium silicate-based material and the type II calcium silicate-based material is stated as followings.

The type I calcium silicate-based material is produced by a sinter method as following steps. Calcium oxide (CaO), silicon dioxide (SiO₂), calcium hydroxide (CaOH₂) and aluminum trioxide (Al₂O₃) are blended by a blender to form a blended oxide. The blended oxide is sintered by a sintering furnace. The blended oxide after sintering has molar ratio of calcium to silicon ranging from 10 to 1. In a first embodiment in accordance with the present invention, a preferred molar ratio of calcium to silicon is ranging from 6 to 2. The blended oxide is cooled and dried to 50° C. to form a type I calcium silicate-based powder after sintered. The blended oxide is dried by an oven under temperature control at −40° C. to 150° C. To avoiding a hydration while grinding the type I calcium silicate-based powder, an ethanol may be added. The calcium silicate-based powder is grinded with the ethanol and dried to form the type I calcium silicate-based material. The preferred concentration of ethanol is 99.5%. In the present embodiment, the calcium silicate-based powder is grinded by a ball miller for 0.5 to 3 days. The calcium silicate-based powder is dried by a oven under temperature control at −40° C. to 100° C. In the present embodiment, the sintering furnace is heated to 900° C. to 1500° C. by increasing temperature at rate of 0.5 to 40° C. per minute. During sintering, process temperature is a constant temperature ranging from 900° C. to 1500° C. about 2 hours. The blended oxide is cooled down to room temperature after sintering. Cooling down means includes but not limited to air blowing, water-cooling or fast cooling techniques with liquid N2 or ice to obtain the type I calcium silicate-based powder.

The type II calcium silicate-based material is produced by a sol-gel method according to followings steps. A tetraethyl orthosilicate (TEOS) is hydrolyzed by a nitric acid solution. A calcium nitrate (Ca(NO3)2) is added into the nitric acid solution to form a type II calcium silicate-based solution. The type II calcium silicate-based solution is dried to form a type II calcium silicate-based powder. The type II calcium silicate-based powder is sintered and grinded to form the type II calcium silicate-based material. The nitric acid solution may break chemical bonds of the tetraethyl orthosilicate (TOES). The broke chemical bonds of the tetraethyl orthosilicate (TOES) may bond with the calcium nitrate (Ca(NO₃)₂) to form the type II calcium silicate-based solution which chemical construction may be a cross-linked network structure. Drying the type II calcium silicate-based material maybe heated the type II calcium silicate-based solution at a constant temperature at 60° C. for 24 hours by an oven and then maintained the temperature at 120° C. to form the type II calcium silicate-based powder. The type II calcium silicate-based powder is sintered at a constant temperature of 500° C. about 2 hours. The type II calcium silicate-based powder is grinded by a ball miller for 12 hours to obtain the type II calcium silicate-based material.

The secondary water or the phosphate solution is blended with the polydopamine modified calcium silicate-based compound in different ratio to form the tiny bone defect repairing material. Physical properties of the tiny bone defect repairing material are as followings.

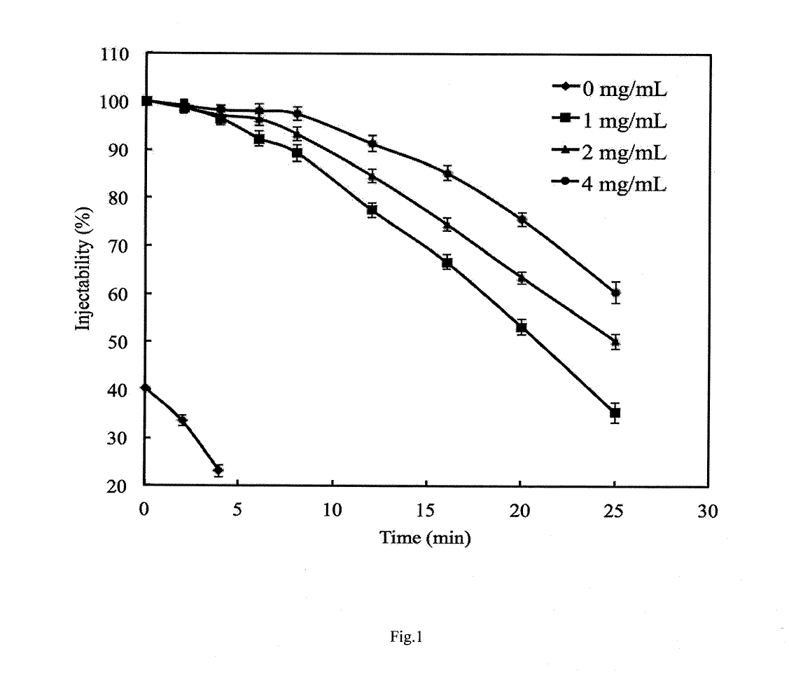

With reference to FIG. 1, abilities of injection of the tiny bone defect repairing material in various blending ratio is showed. The testing method has steps of blending the secondary water or the phosphate solution with the polydopamine modified calcium silicate-based compound in different ratio to form the tiny bone defect repairing material. The different ratio of the tiny bone defect repairing material is infused to a syringe where a capacity of each syringe is 5 mL. A diameter of the needle opening of each syringe is 2.0 mm. The syringe is being put into a hydrate environment. The hydrate environment is at temperature of 37° C. and under humidity of 100%. The tiny bone defect repairing material is extruded from the syringe in different time period until the tiny bone defect repairing material is unable to extruded form the syringe. In one embodiment, the tiny bone defect repairing material in blending ratio of 0 mg/mL is only infused the polydopamine modified calcium silicate-based compound in to the syringe. The setting time of the tiny bone defect repairing material in blending ratio of 0 mg/mL is under 5 minutes which causes a poor injectability. The injectability of the tiny bone defect repairing material is increasing as the blending ratio increased. Normally, the tiny bone defect repairing material is clinical usable since 20 minutes injectability thereof after being infused in to the syringe is available. In other embodiments of the present invention, the tiny bone defect repairing material with blending ratio of 1 to 4 mg/mL still remain 50% of injectability which means the present invention is able to repair tiny bone defects by injected with the syringe which significantly increasing the possibility in clinical use. Injectability is defined as the percentage by volume of the amount of the tiny bone defect repairing material that could be extruded from the syringe with the total volume of the tiny bone defect repairing material in the syringe. In these embodiments, at least 2.5 mL of the tiny bone defect repairing material with blending ratio of 1 to 4 mg/mL in the syringe (the total capacity of each syringe is 5 mL) are able to extruded form the syringe with 20 minutes.

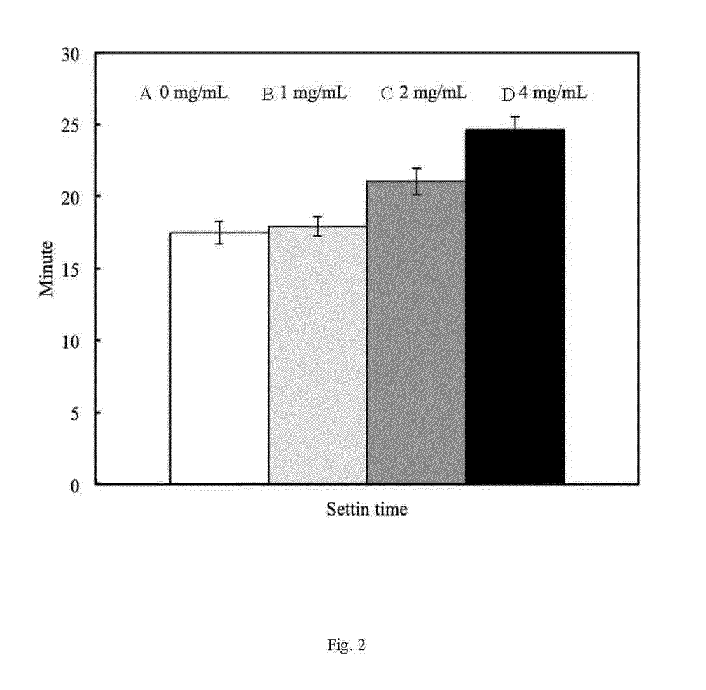

With reference to FIG. 2, a setting time of the artificial material in different blending ratio is disclosed. The secondary water or the phosphate solution is blended with the polydopamine modified calcium silicate-based compound in different ratio to form the tiny bone defect repairing material. The tiny bone defect repairing material is filled into a mould to form a testing block and then the mould is preserved in a hydrate environment. Each mould has a diameter of 6 mm and a height of 3 mm. Temperature of the hydrate environment is controlled at 37° C. Relative humidity of the hydrate environment is 100%.

The tiny bone defect repairing material is removed from the mould after hydration reaction is completed to from a tiny bone defect repairing material testing block. The setting time of the tiny bone defect repairing material testing block is measured. A testing standard of setting time used in the accordance with the present invention is ASTM C 187-98 (American Society for Testing and Materials). Normally, setting time of artificial bone repairing materials should be less than 40 minutes for clinical use. In the present embodiment, the setting times of the present invention are all less than 30 minutes, thus the embodiments in accordance with the present invention is proven being suitable for the clinical use.

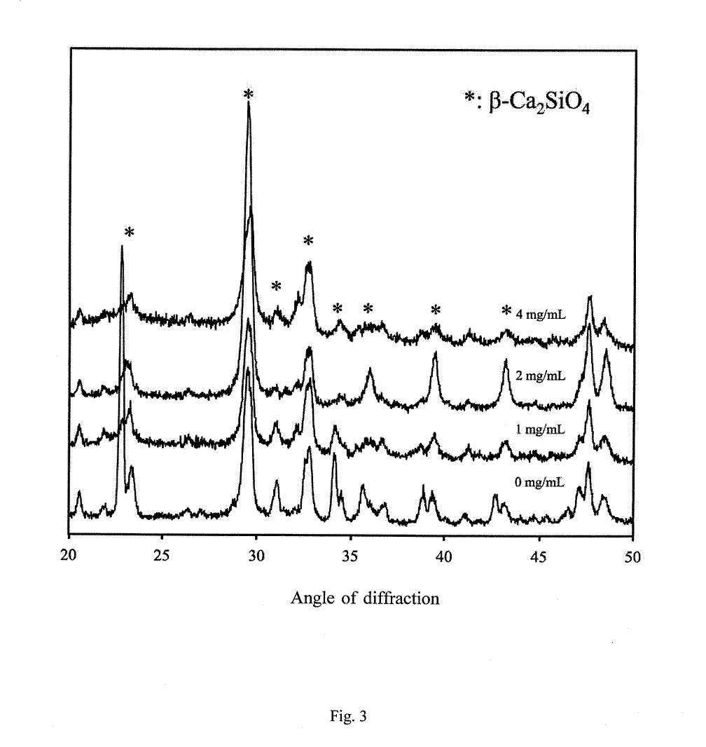

The above mentioned tiny bone defect repairing material testing block is put into the hydrate environment again for a day. Putting the bone defect repairing material testing block into the hydrate environment again is capable of increasing the intensity of the bone defect repairing material. With reference to FIG. 3, XRD patterns showed main constituent of the polydopamine modified calcium silicate-based compound is β-phase calcium silicate (β-Ca2SiO4).

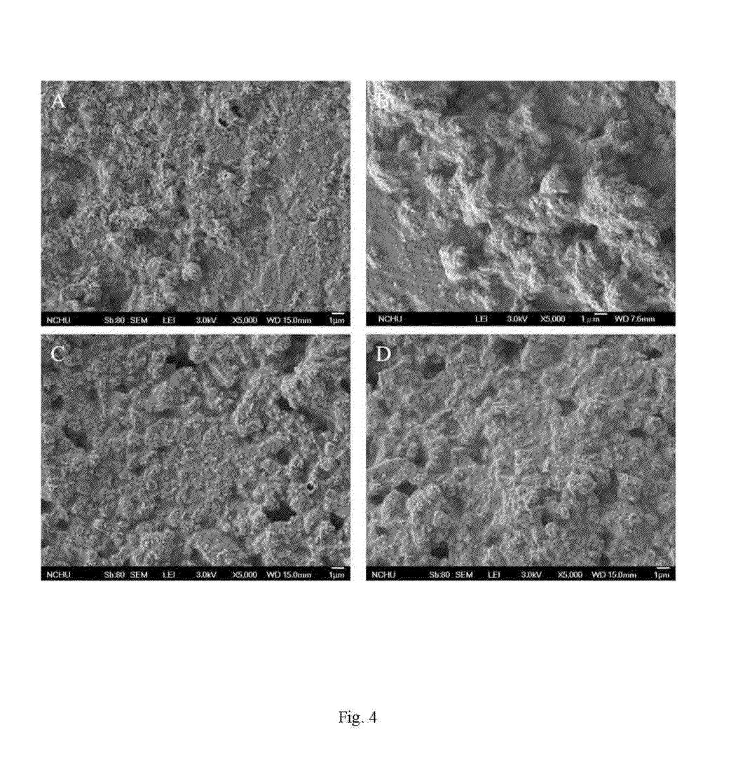

The tiny bone defect repairing material testing block is put into a stimulated body fluid (SBF) 10 mL for a period of time and the tiny bone defect repairing material testing block is then dried by an oven. A mass loss of the tiny bone defect repairing material testing block and strength of surface structure of the tiny bone defect repairing material testing block are observed by an electronic microscope. With reference to FIG. 4, sample A represents the blending ratio of the tiny bone defect repairing material testing block is 0 mg/mL, the blending ratio of the tiny bone defect repairing material testing block of sample B is 1 mg/mL, the blending ratio of the tiny bone defect repairing material testing block of sample C is 2 mg/mL and the blending ratio of the tiny bone defect repairing material testing block of sample D is 4 mg/mL. According to FIG. 4, the surface structures of sample A, B, C and D have no damage.

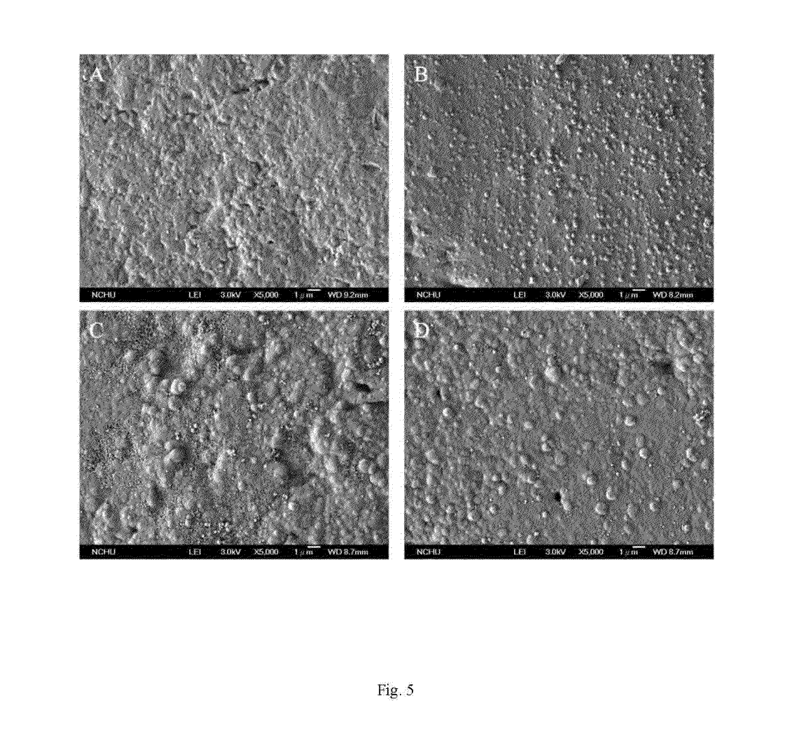

The above mentioned testing block is put into a stimulated body fluid (SBF) 10 mL for a period of time and a growth of apatite on the tiny bone defect repairing material testing block is observed by an electronic microscope. With reference to FIG. 5, the growths of apatite of sample A, B, C and D are plenty which means the present invention has great bioactivity.

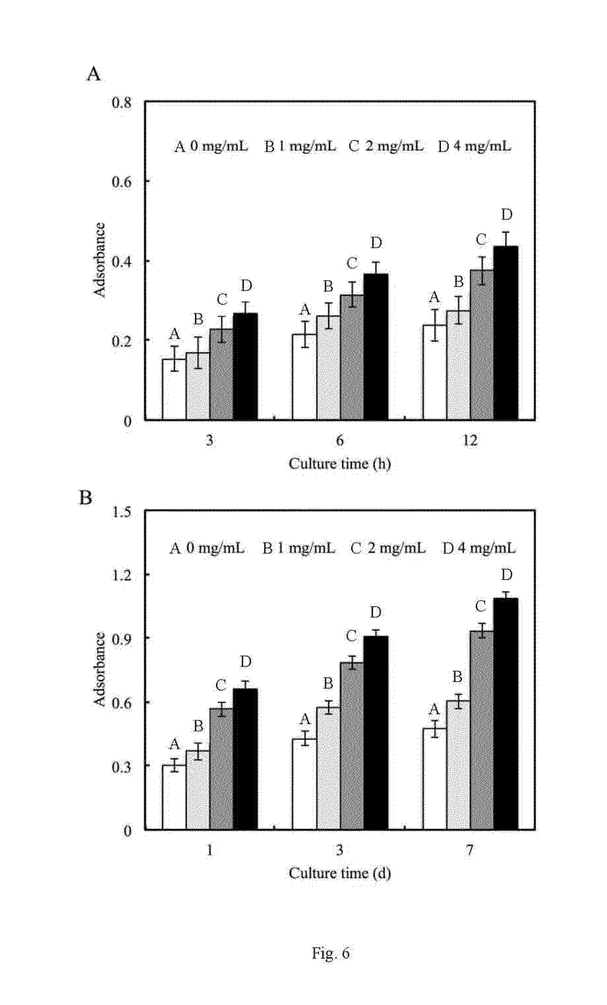

The testing method of the present invention has no cytotoxicity and is able to stimulate cells to attach and grow having steps of:
sterilizing the polydopamine modified calcium silicate-based compound by a 75% ethanol solution; exposing the polydopamine modified calcium silicate-based compound to a UV light for an hour; culturing primary human pulp fibroblast (HPF) on the sterilized polydopamine modified calcium silicate-based compound. A growth rate of the primary human pulp fibroblast (HPF) is observed in different timing. With reference to FIG. 6, the growth rate of the primary human pulp fibroblast (HPF) is increased according to content of the polydopamine in the polydopamine modified calcium silicate-based compound.

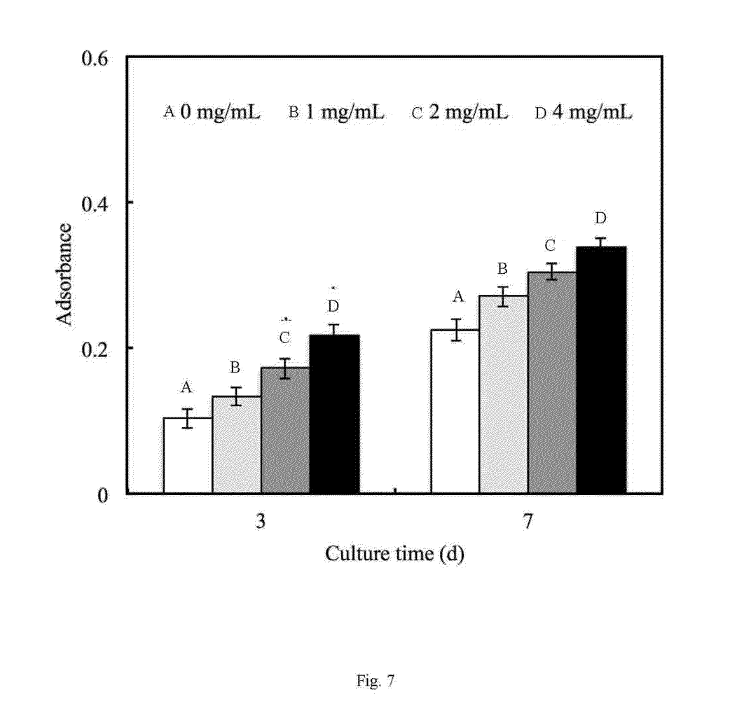

An activity of phosphatase and a production of osteocalcin are an important index for bone cell differentiation. The primary human pulp fibroblast (HPF) is cultured on the sterilized polydopamine modified calcium silicate-based compound. The activity of phosphatase and the production of osteocalcin of the primary human pulp fibroblast (HPF) are observed in different time. With reference to FIG. 7, the activity of phosphatase and the production of osteocalcin of the primary human pulp fibroblast (HPF) are increased when the content of the polydopamine in the polydopamine modified calcium silicate-based compound are increased, which means the present invention is able to stimulate cell differentiation.

According to aforementioned result, the preferred ratio of the secondary water or the phosphate solution to the polydopamine modified calcium silicate-based compound is between 0.3˜1.5 mL/g.

Thus, the present invention achieves advantages as below. 1. Providing a tiny bone defect repairing material with high performance in abilities of injection, plasticity for clinical use. 2. The present invention provide high standard of mechanical property, bioactivity and clinical used property of the tiny bond defect repairing material. 3. The present invention provides improved bioactivity and water absorbs ability compared to the conventional calcium silicate-based bone cement.

What is claimed is:

1. A producing method of a tiny bone defect repairing material having steps of:
mixing a polydopamine solution with a calcium silicate-based material to form a polydopamine modified calcium silicate-based compound; and
blending a secondary water or a phosphate solution with the polydopamine modified calcium silicate-based compound to obtain the tiny bone defect repairing material.

2. The producing method of the tiny bone defect repairing material as claimed in claim 1, wherein the calcium silicate-based material is mixed with the polydopamine solution by stirring and the calcium silicate-based material and the polydopamine solution are filtered to form a mixing powder;
using a secondary water to wash the mixing powder; and
filtering the mixing powder and drying the mixing powder to form the polydopamine modified calcium silicate-based compound.

3. The producing method of the tiny bone defect repairing material as claimed in claim 1, the calcium silicate-based material is a type I calcium silicate-based material, the type I calcium silicate-based material is prepared by steps of
blending calcium oxide, silicon dioxide, calcium hydroxide and aluminum trioxide to form a blended oxide;
sintering the blended oxide and cooling the blended oxide to form a type I calcium silicate-based powder; and
grinding the calcium silicate-based powder with ethanol to form the type I calcium silicate-based material.

4. The producing method of the tiny bone defect repairing material as claimed in claim 2, the calcium silicate-based material is a type I calcium silicate-based material, the type I calcium silicate-based material is prepared by steps of
blending calcium oxide, silicon dioxide, calcium hydroxide and aluminum trioxide to form a blended oxide;
sintering the blended oxide and cooling the blended oxide to form a type I calcium silicate-based powder; and
grinding the calcium silicate-based powder with an ethanol to form the type I calcium silicate-based material.

5. The producing method of the tiny bone defect repairing material as claimed in claim 1, the calcium silicate-based material is a type II calcium silicate-based material, wherein
a tetraethyl orthosilicate is hydrolyzed with a nitric acid solution and a calcium nitrate is added to form a type II calcium silicate-based solution;
the type II calcium silicate-based solution is dried to form a type II calcium silicate-based powder; and
the type II calcium silicate-based powder is sintered and grinded to form the type II calcium silicate-based material.

6. The producing method of the tiny bone defect repairing material as claimed in claim 2, the calcium silicate-based material is a type II calcium silicate-based material, wherein
a tetraethyl orthosilicate is hydrolyzed with a nitric acid solution and a calcium nitrate is added to form a type II calcium silicate-based solution;
the type II calcium silicate-based solution is dried to form a type II calcium silicate-based powder; and
the type II calcium silicate-based powder is sintered and grinded to form the type II calcium silicate-based material.

7. The producing method of the tiny bone defect repairing material as claimed in claim 3, wherein
the calcium oxide, the silicon dioxide, the calcium hydroxide and the aluminum trioxide are blended by a blender for 0.5 to 2 hours;
the blended oxide is sintered by a sintering furnace which the sintering furnace heats to 900° C. to 1500° C.;
the blended oxide is cooled to room temperature by air-cooling or water-cooling;
the calcium silicate-based powder is grinded for 0.5 to 3 days to form the type I calcium silicate-based material.

8. The producing method of the tiny bone defect repairing material as claimed in claim 4, wherein
the calcium oxide, the silicon dioxide, the calcium hydroxide and the aluminum trioxide are blended by a blender for 0.5 to 2 hours;
the blended oxide is sintered by a sintering furnace which the sintering furnace is heated to 900° C. to 1500° C.;
the blended oxide is cooled to room temperature by air-cooling or water-cooling;
the calcium silicate-based powder is grinded for 0.5 to 3 days to form the type I calcium silicate-based material.

9. The producing method of the tiny bone defect repairing material as claimed in claim 5, wherein the type II calcium silicate-based solution is dried at a constant temperature of 60° C. for a day by an oven and maintained at a temperature of 120° C. to form the type II calcium silicate-based powder;
the type II calcium silicate-based powder is sintered at a constant temperature of 500° C. for 2 hours by a sinter; and
the type II calcium silicate-based powder is grinded by a ball miller for 12 hours to obtain the type II calcium silicate-based material.

10. The producing method of the tiny bone defect repairing material as claimed in claim 6, wherein the type II calcium silicate-based solution is dried at a constant temperature of 60° C. for a day by an oven and maintained at a temperature of 120° C. to form the type II calcium silicate-based powder;
the type II calcium silicate-based powder is sintered at a constant temperature of 500° C. for 2 hours by a sinter; and
the type II calcium silicate-based powder is grinded with a ball miller for 12 hours to obtain the type II calcium silicate-based material.

11. A tiny bone defect repairing material having a secondary water or a phosphate solution being uniformly blended with a polydopamine modified calcium silicate-based compound, wherein
the polydopamine modified calcium silicate-based compound is a mixture of a polydopamine solution and a calcium silicate-based material; and
a main constituent of the calcium silicate-based material in the polydopamine modified calcium silicate-based compound has β-phase calcium silicate.

12. The tiny bone defect repairing material as claimed in claim 11, wherein the polydopamine modified calcium silicate-based compound is produced by a sinter method having steps of:
blending calcium oxide, silicon dioxide, calcium hydroxide and aluminum trioxide to form a blended oxide;
sintering the blended oxide; and
mixing the polydopamine solution with the calcium silicate-based material to form the polydopamine modified calcium silicate-based compound.

13. The tiny bone defect repairing material as claimed in claim 11, wherein
the blended oxide has a molar ratio of calcium to silicon ranging from 10 to 1; and
a ratio of the secondary water or the phosphate solution to the polydopamine modified calcium silicate-based compound is between 0.2˜4 mL/g.

14. The tiny bone defect repairing material as claimed in claim 11, wherein
the blended oxide has a molar ratio of calcium to silicon ranging from 6 to 2;
a ratio of the secondary water or the phosphate solution to the polydopamine modified calcium silicate-based compound is between 0.3˜1.5 mL/g;
the polydopamine solution is to dissolve a polydopamine powder into water to form a polydopamine liquid;
the polydopamine liquid is mixed with a Tris buffer solution, where pH value of the Tris solution is 8.55 and temperature of the Tris buffer solution is 80° C.; and
the concentration of the polydopamine liquid is 0.5˜20 mg/mL.

15. The tiny bone defect repairing material as claimed in claim 14, wherein
the concentration of the polydopamine liquid is 0.5˜5.0 mg/mL; and
the ratio of the secondary water or the phosphate solution to the polydopamine modified calcium silicate-based compound is between 0.8˜1.5 mL/g.

16. A matrix material of a tiny bone defect repairing material, wherein
a polydopamine modified calcium silicate-based compound is to mix a polydopamine solution with a calcium silicate-based material; and
the main constituent of the calcium silicate-based material is β-phase calcium silicate.

17. The matrix material of the tiny bone defect repairing material as claimed in claim 16, wherein
sintering a blended oxide to form the calcium silicate-based material; and
the blended oxide has a molar ratio of calcium to silicon ranging from 10 to 1.

18. The matrix material of the tiny bone defect repairing material as claimed in claim 17, wherein the blended oxide has a molar ratio of calcium to silicon ranging from 6 to 2.
